(19) United States
(12) Patent Application Publication
(10) Pub. No.: US 2013/0211105 A1
(43) Pub. Date: Aug. 15, 2013

(54) ACYLATIONS IN MICRO REACTION SYSTEMS

(71) Applicants: Werner Bonrath, Basel (CH); Ingo Koschinski, Basel (CH); Thomas Van Oordt, Basel (CH)

(72) Inventors: Werner Bonrath, Basel (CH); Ingo Koschinski, Basel (CH); Thomas Van Oordt, Basel (CH)

(21) Appl. No.: 13/521,498

(22) PCT Filed: Jan. 13, 2011
(86) PCT No.: PCT/EP11/50412

§ 371 Date: Nov. 20, 2012

(30) Foreign Application Priority Data
Jan. 13, 2010 (EP) 10150672.3

(51) Int. Cl. C07C 67/08; C07D 311/58

(52) U.S. Cl. CPC C07C 67/08; C07D 311/58
USPC 549/410; 560/265; 560/261

(57) ABSTRACT

A method for acylating tertiary alcohols and phenolic compounds with carboxylic acids or their anhydrides in micro-reaction systems wherein the acylation is effected in the absence of any catalyst including water at residence times of at most 30 minutes.

The present invention relates to a method for acylating tertiary alcohols and phenolic compounds in modular micro-reaction systems.

Acylations of alcohols, especially acetylations, are among the most important reactions in organic chemistry and useful in the preparation of commercially valuable products, e.g., pharmaceuticals, agrochemicals or flavors, and intermediates therefore.

On the one hand acylations of organic hydroxy compounds can be carried out by reacting the hydroxy compound with an acid. Better yields are normally achieved if acid derivatives are used, e.g., acid anhydrides or acid halogenides. On the other hand, in order to achieve good yields, catalysts are used, mainly acidic catalysts, which, however, may give rise to undesired side reactions, e.g., elimination of water from tertiary alcohols, or attacks of centers of asymmetry, thus influencing stereochemistry unfavorably. Basic catalysts which do not show these disadvantages are normally less effective because of longer reaction times.

It is an object of the present invention to provide a commercially attractive method for acylating organic hydroxy compounds, more precisely, of tertiary alcohols and phenolic compound with acids or their anhydrides without using any catalysts.

During the last decade the miniaturization of chemical reactors has offered many fundamental and practical advantages of relevance to the chemical industry and has been developed to an extent that methods of using micro-reactors in chemical synthesis are applicable not only at laboratory scale but for the production of commercially important amounts. It has been demonstrated that chemical syntheses in micro-reactors are broadly applicable and syntheses by many different reaction types in different micro-reactors and micro-reactor systems, particularly in modular reaction systems, have been successfully realized and are described in the literature; see, e.g., P. D. I. Fletcher et al., Tetrahedron 58, 4735-4755 (2002); W. Ehrfeld et al. in Ullmann's Encyclopedia of Industrial Chemistry, 6^{th} edition, 1999; and V. Hessel et al., Angew. Chemie, Int. Ed., 43, 406-451 (2004) which are all introduced herein by reference.

T. Schwalbe et al. in Chimia 56, 636 ff (2002) describe the acylation in micro-reactors of several amines of the general formula R—CH₂—NH₂ with Ac₂O/Et₃N in DMF or dioxane with yields of up to 100% at residence times of from 1 to 13 minutes and throughputs of from 6.1 to 68.3 g/h. D. A. Snyder et al. in Helv. Chimica Acta 88, 1-9 (2005), have described the production of 2-phenylethyl acetate from 2-phenyl ethanol using excess of Ac₂O and 4-(dimethylamino)-pyridine (DMAP) as catalyst in a modular micro-reaction system. Nowhere the acylation of organic hydroxy compounds in micro-reaction systems has been described with acids and in the absence of catalysts.

Recently, Sato et al. in Angew. Chem. Int. Ed. 46, 6284-8, 2007, have described a highly efficient acylation of alcohols and phenols with acetic anhydride without acid or base catalysts that involves micro-reaction system with subcritical water as both the catalyst and the substrate and product phase. The authors suggest that their results support the ability of subcritical water to act as a Lewis acid. Lewis acids are known catalysts in acylations. The desired esters are obtained in excellent yield with high selectivity at 200° C. In a typical procedure a stream containing a mixture of alcohol and anhydride is placed across a high-speed flow of subcritical water and the resulting mixture is introduced into micro-reactor, where the acylation proceeds rapidly without significant side-reactions. The product accumulates at the bottom of the aqueous solution and can be easily and quantitatively isolated by phase separation or filtration.

E. Bulychev in Pharmaceutical Chemistry Journal 32, 331-2 (1998) points to the fact that introduction of the acetyl group into the molecule of tocopherol markedly increases its stability with respect to long-term storage and oxidation, while not affecting the physiological activity. On the other hand the maximum allowable percentages of alpha-tocopherol in the commercial vitamin E acetate, as stipulated by the pharmacopoeias of various countries vary from 0.5% to 3.0%. An excess content of free alpha-tocopherol in the final commercial alpha-tocopherol acetate reduces its quality and decreases the maximum storage duration. This illustrates that there is a need for a commercial production process of alpha-tocopherol acetate which produces the desired product in high purity, in high yield in as short a time as possible. The alpha-tocopherol acetylation is a fast reaction and virtually irreversible under normal conditions, e.g., with acetic anhydride, a constant concentration of catalyst (sulfuric acid), at temperatures of 60, 80 and 100 ° C. At higher temperatures, however, the reaction becomes reversible which leads to higher concentrations of alpha-tocopherol in the desired end product. Therefore, the reaction time must be sufficiently short to avoid establishing an undesired equilibrium with relatively high percentages of alpha-tocopherol as side products.

Acetylation of alpha-tocopherol with acetic acid anhydride in the presence of various catalysts is well-known and documented. EP 0 784 042 A1, published Jul. 16, 1997, describes this reaction wherein hydrogen bis(oxalate)borate is used as the catalyst. After heating to reflux for one hour crude d,l-alpha-tocopherol was obtained in 92% yield which had a content of 87%.

KR 10-2001-0090181, published 18.10.2001, discloses a method for preparation of high yield, high purity D,L-alpha-tocopherol-acetate, wherein the reactants consisting of D,L-alpha-tocopherol and acetic anhydride are fed into a continuous tubular reactor and reacted in the absence of a catalyst at 139-250° C. and 2-20 atm. In accordance with the two Examples given, a bead-filled tubular reactor with a volume of 130 ml was used and a mixture of 1 kg and 2 kg of DL-alpha-tocopherol, respectively, with 500 g of acetic anhydride was fed to the reactor at a rate of 100 ml/hour and a temperature of 205° C. and 250° C., respectively, of the reactor. Conversion rates of 99.6% and 99.3%, respectively, are reported. However, nothing is said about the selectivity of the reaction, i.e. the purity of the alpha-tocopherol acetate and the impurities/side products. Due to the lack of details in the description of the experiments they could not be repeated.

In an attempt to further improve this micro-reaction method of acylating alcohols and phenols it has been found in accordance with the present invention that similar excellent results in the acylation of tertiary alcohols and phenolic compounds in micro-reaction systems are obtained in the absence of any catalysts including water as catalyst and carrier. Thus, since the elimination of major amounts of water from the reaction mixture becomes unnecessary energy is saved and makes the present process commercially more attractive.

The present invention, therefore, relates to a method of acylating tertiary alcohols and phenolic compounds with carboxylic acids or their anhydrides in micro-reaction systems which method is characterized in that it is effected in the absence of any catalyst including water at a residence time of at most 30 minutes.

In connection with the present invention the terms micro-reactions and micro-reaction systems apply to chemical micro-processing in its broadest sense as described in the state of the art and which is generally defined as continuous flow through regular domains in which the internal structures of fluid channels have characteristic dimensions, typically in the “sub-millimeter” range (Hessel, V. et al., Chemical Microprocess Engineering: Fundamentals, Modelling and Reactions, Wiley-VCH, Weinheim, 2004). However, systems wherein the inner diameters of the fluid channels are in the millimeter dimension, i.e. from 1-5 mm, preferably 1, 2 or 3 mm, can also be successfully used with good results. In a preferred embodiment modular micro-reaction systems are used thereby taking advantage of the known general advantages modular systems provide.

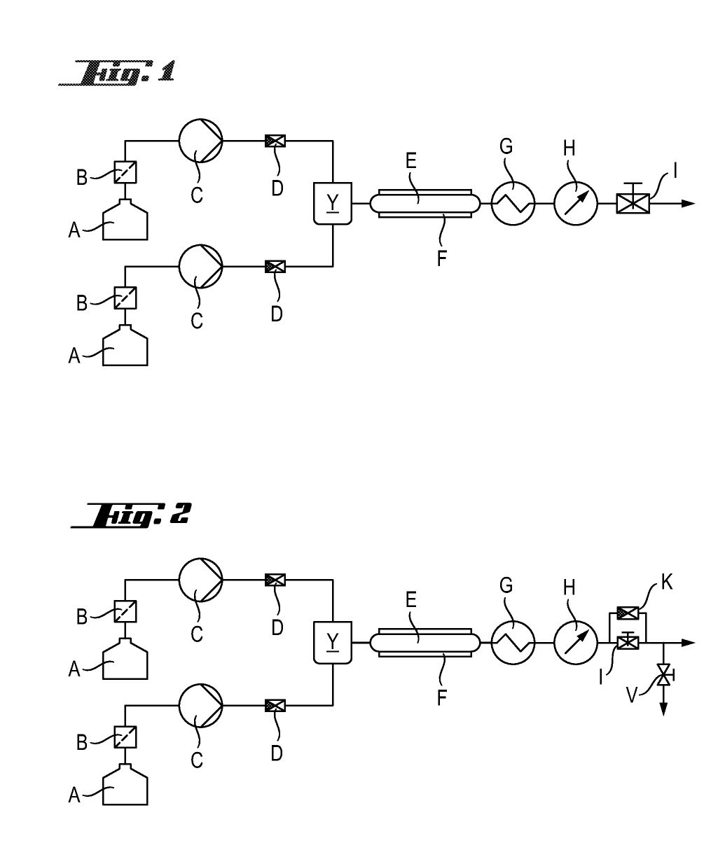

FIGS. 1 and 2 describe in general micro-reaction systems which can be used in the present invention and which comprise the containers (A) with the reactants (alcohol or phenol and acylating agent, respectively), filters (B), pumps (C), non-return valves (D), a mixing unit, e.g., T-piece (Y), micro-reactor (E), oil bath or heating jacket (F), cooling element (G), pressure gauge (H), needle valve (I) non-return valve (K) and sampling valve (V).

The reaction mixture is then worked up by methods well-known in the art.

The terms “tertiary alcohols” and “phenolic compounds” are used herein in their broadest usual sense and cover all such compounds having a hydroxy group which is amenable to acylation. The aliphatic chain of a tertiary alcohol may be a straight- or branched-chain, possibly cyclic, saturated or unsaturated, i.e., with one or more carbon-carbon double and/or triple bond(s), and substituted with one or more substituents resistant to modification under the reaction conditions. The phenolic compounds, viz. aromatic alcohols, may be carbocyclic and/or hetero-cyclic compounds of monocyclic or condensed nature, viz. may contain two, three or more cycles. The hydroxy compounds may have preferably 1-50 carbon atoms. Examples of unsaturated tertiary alcohols are nerol, linalool, dehydrolinalool, nerolidol and isophytol. Of specific interest within this group are those compounds which have applications as flavorings or fragrances and are parts of perfumes, among which are many mono- and bicyclic monoterpenes (C10-compounds), e.g., terpineols; and phenols, e.g., thymol (or p-cymenol). Within the group of terpenoid or isoprenoid compounds there are tertiary alcohols belonging to the sesquiterpenes (C15), diterpenes (C20), triterpenes (C30) and tetraterpenes (C40). Representatives of triterpenes are calciferols and of tetraterpenes are carotenoids. Also covered by the above definition are isoprenoid tertiary alcohols with more than 4 isoprenyl residues, i.e., having 25, 30, 35, 40, 45, 50, etc., carbon atoms, known as polyprenols. A group of “phenolic compounds” of specific interest within the present invention are tocopherols. The term “tocopherol” as used herein is to be understood to refer to tocol and any compound derived from the basic structure of tocol [2-methyl-2-(4′,8′,12′-trimethyltridecyI)-6-chromanol], having a free 6-hydroxy group and exhibiting vitamin E activity, viz. any tocopherol having the saturated side chain 4′,8′,12′-trimethyltridecyl, such as α-, β-, γ-, δ-, ζ₂- or η-tocopherol, and also any tocotrienol having three double bonds in the side chain [4′,8′,12′-trimethyltridec-3′,7′,11′-trienyl], such as ε- or ζ₁-tocopherol. Of these various tocopherols (all-rac)-α-tocopherol, generally referred to as vitamin E, is of primary interest, being the most active and industrially most important member of the vitamin E group.

The acylation can be carried out with aliphatic and aromatic mono-, di- and poly-carboxylic acids and/or their corresponding anhydrides which are liquid under the reaction conditions thus avoiding the use of solvents. Aliphatic acids, preferably C₁₋₈ saturated acids, may be branched- or straight-chain, such as formic acid, acetic acid, propionic acid, isopentanoic acid, preferably acetic acid, and representatives of aromatic acids are benzoic acid, phthalic acid and gallic acid. The most preferred acylating agent is acetic acid anhydride.

The acylations of the present invention can conveniently be carried out in a temperature range of from 80-280° C., preferably 100-250° C., under a pressure sufficient to prevent boiling of the reaction mixture which is normally in the range of from 6-50 bar, preferably 6-35 bar. However, these parameters can be changed according to the circumstances. The dimensions of the micro-reaction system used in the present invention can also vary within broad limits and be adapted to the requirements. The molar ratio of hydroxy compound:acylating agent can vary in the range of from 1:1 to 1:10 and is preferably in the range of 1:1-5. Most preferably only a slight excess of acylating agent is used, e.g., 1.2-1.5:1 mol/mol.

The acylations can be carried out without a solvent or with inert solvents from which the desired product can be easily isolated and, if necessary, purified.

In most cases the reaction is completed with high yields and high selectivity at a residence time of the reactants in the reactor of at most 30 minutes, preferably at shorter residence times, e.g., of 20, 15, 10 or less than 10 minutes. On the other hand longer residence times may be necessary to achieve the desired results, depending of the dimensions of the equipment.

### Equipment:

Merck Hitachi L600 and L6200 HPLC-piston pump (0-10 ml/min.) including filter 638-1423.

Back-pressure valve Nupro/Swagelok (1 PSI).

Mixing unit (external oil bath): Swagelok 1/16 inchT-piece.

Residence time: 45 ml steel pipe (1.4435 steel, 3 mm inner diameter) located into oil bath, heat exchanger Ehrfeld-Komponente (300 μm, 0309-2-0001-F).

Pressure measurement: WIKA (S-11, 0-100 bar).

Needle valve Swagelok ⅛ inch.

Non-return valve Swagelok ⅛ inch (30 bar)

Sampling valve Swagelok ⅛ inch

### General Procedure:

The alcohol or phenol/acetic anhydride or acetic acid mixture (premixed at room temperature (1.0:1.2 mol) was pumped using HPLC pumps with a discharge pressure of 40 bar into the stainless steel tube which was heated in an oil bath to the required process temperature. The reaction mixture was then quenched to room temperature using a micro heat exchanger. The pressure of the cooled down reaction mixture was reduced using a pressure control valve. The reaction mixture was analyzed by GC and the concentrations of alcohol/phenol and corresponding ester were measured.

### EXAMPLES and RESULTS

### Example 1

Acetylation of tert.-butanol with acetic acid anhydride (1.0:1.2 mol) without catalyst; 30 bar. The microreactor system used was that shown in FIG. 1.

The results of the reaction at different temperatures and different residence times are given in Table 1 below.

TABLE 1

| Temperature | Residence time | Conversion | Yield tert.-butyl |
|---|---|---|---|
| ° C. | min | tert.-butanol % | acetate % |
| 175 | 5 | 64 | 77 |
| 175 | 10 | 71 | 96 |
| 175 | 20 | 60 | 99 |
| 200 | 2.5 | 72 | 94 |
| 200 | 5 | 59 | 97 |
| 200 | 10 | 17 | 100 |
| 150 | 2.5 | 35 | 34 |
| 150 | 16.8 | 79 | 88 |

Analysis of reaction mixture by GC method:

| Instrument: | Perkin Elmer Autosystem XL with Split-Injector | |
|---|---|---|
| | and FID | |
| Column: | Stationary phase: | HP-5-column (crosslinked 5% |
| | | PH ME Siloxane) |
| | Length × ID: | 30 m × 0.53 mm; |
| | | Film 2.65 μm |
| Carrier Gas: | Gas: | Helium |
| | Mode: | constant flow 4 ml/min |
| Oven program: | 55° C. (5.5 min) → 12° C./min → 90° C. → | |
| | 25° C./min → 270° C. | |
| Injector temperature: | 250° C. | |
| Injection volume: | 0.5 μl | Split ratio 1:10 |
| Detector temperature: | 250° C. | |

In Examples 2 to 4, the set-up of the microreactor system was slightly modified as depicted in FIG. 2.

### Example 2

Acetylation of d,l-α-tocopherol with acetic acid anhydride (1.0:1.1 mol) without catalyst; 30 bar.

The same equipment as depicted in FIG. 2 was used with the exception that only one pump was used to pump the reaction mixture which was premixed at room temperature, via the mixer into the residence tube.

The results of the reaction at different temperatures and different residence times are given in Table 2 below.

TABLE 2

| Temperature | Residence time | Conversion | Yield tocopherol |
|---|---|---|---|
| ° C. | min | tocopherol % | acetate % |
| 150 | 21.8 | 45.7 | 45.1 |
| 150 | 32.9 | 49.8 | 48.8 |
| 150 | 42.8 | 62.2 | 61.5 |
| 200 | 21.8 | 93.9 | 91.9 |
| 200 | 32.9 | 95.6 | 92.9 |
| 200 | 42.8 | 98.0 | 96.6 |
| 220 | 21.8 | 97.4 | 94.6 |
| 220 | 32.9 | 99.4 | 94.4 |
| 220 | 42.8 | 99.7 | 97.4 |
| 240 | 21.8 | 99.9 | 97.0 |
| 240 | 32.9 | 99.4 | 94.9 |
| 240 | 12 | 99.2 | 97.8 |
| 250 | 12 | 99.6 | 97.6 |
| 250 | 21.8 | 99.8 | 97.7 |

Analysis of reaction mixture was done by GC method:

| Instrument: | Agilent Technologies 6890 N with Split-Injector | |
|---|---|---|
| | and FID | |
| Column: | Stationary phase: | CP-SIL 8 CB, Varian, Cat. |
| | | No. CP 7761 |
| | Length × ID: | 25 m × 0.32 mm; Film 1.2 μm |
| Carrier Gas: | Gas: | Helium |
| | Mode: | constant pressure 18 psi |
| Oven program: | 300° C. (25 min) | |
| Injector temperature: | 300° C. | |
| Injection volume: | 1 μl | Split ratio 1:40 |
| Detector temperature: | 280° C. | |

### Example 3

Acetylation of dehydrolinalool (3,7-dimethyl-6-octen-1-yn-3-ol) with acetic acid anhydride (1.0:1.2 mol) without catalyst; 30 bar.

The same equipment as described in Example 2 was used for the experiments.

The results of the reaction at different temperatures and different residence times are given in Table 3 below.

TABLE 3

| Temperature | Residence time | Conversion de- | Yield dehydrolinlalyl |
|---|---|---|---|
| ° C. | min | hydrolinalool % | acetate % |
| 200 | 5.5 | 73.1 | 58.1 |
| 200 | 10.8 | 90.1 | 61.7 |
| 200 | 21.8 | 98.8 | 47.1 |
| 160 | 10.8 | 33.3 | 29.8 |
| 160 | 21.8 | 54.2 | 47.1 |
| 160 | 43.9 | 75.7 | 63.0 |
| 150 | 10.8 | 20.6 | 18.9 |
| 150 | 21.8 | 37.3 | 32.8 |
| 150 | 43.9 | 59.1 | 50.7 |
| 120 | 43.9 | 13.8 | 13.2 |

Analysis of the reaction mixture was done by GC method:

| Instrument: | Agilent Technologies 6890 N with Split-Injector | |
|---|---|---|
| | and FID | |
| Column: | Stationary Phase: | Optima delta 3 Macherey- |
| | | Nagel Cat. No. 726442.30 |
| | Length × ID: | 30 m × 0.32 mm; Film 1.0 μm |
| Carrier Gas: | Gas: | Helium |
| | Mode: | constant pressure 18 psi |
| Oven program: | 60° C. (0 min) → 6° C./min → 120° C. → | |
| | 10° C./min→ 300° C. (5 min) | |
| Injector temperature: | 250° C. | |
| Injection volume: | 1 μl | Split ratio 1:40 |
| Detector temperature: | 280° C. | |

### Example 4

Acetylation of d,l-α-tocopherol with acetic acid (1.0:2.0 mol) without catalyst; 30 bar.

The same equipment as in Example 2 was used in the experiments.

The results of the reaction at different temperatures and different residence times are given in Table 4 below.

Analysis was done by GC method:

| Instrument: | HP 6890 with Split-Injector and FID | |
|---|---|---|
| Column: | Stationary Phase: | Rtx-5SilMS (Cat# 12794) |
| | Length × ID: | 30 m × 0.28 mm; Film 0.5 μm |
| Carrier Gas: | Type: | Helium |
| | Mode: | constant flow 1.5 ml/min |
| Oven program: | 150° C. (0 min) → 5° C./min → 335° C. (8 min) | |
| Injector temperature: | 300° C. | |
| Injection volume: | 1 μl | Split ratio 1:50 |
| Detector temperature: | 330° C. | |

TABLE 4

| Temperature | Residence time | Conversion | Yield tocopherol |
|---|---|---|---|
| ° C. | min | tocopherol % | acetate % |
| 250 | 30 | 12.3 | 12.3 |
| 250 | 60 | 18.8 | 17.7 |

Although the yields are lower than in case of acetylation with acetic acid anhydride the results are attractive for commercial production in view of the difficulties and disadvantages of this reaction in normal reactors.

1. A method for acylating tertiary alcohols and phenolic compounds with carboxylic acids or their anhydrides in micro-reaction systems characterized in that the acylation is effected in the absence of any catalyst including water at a residence time of at most 30 minutes.

2. The method of claim 1, wherein the micro-reaction system is a modular micro-reaction system.

3. The method of claim 1, wherein the tertiary alcohol is an aliphatic or araliphatic alcohol.

4. The method of claim 1, wherein the acylation is effected with an acid anhydride, particularly with acetic acid anhydride.

5. The method of claim 1, wherein the tertiary alcohol is an allylic alcohol, particularly linalool, dehydrolinalool, nerolidol or isophytol.

6. The method of claim 1, wherein the phenolic compound is a tocopherol or tocotrienol, particularly d,l-alpha-tocopherol.

7. The method of claim 1, wherein the acylation is effected at a temperature in the range of 80-280° C., preferably of 100-250° C.

8. The method of claim 1, wherein the acylation is effected under a pressure sufficient to prevent boiling of the reaction mixture.
